(19) United States
(12) Patent Application Publication
(10) Pub. No.: US 2012/0009264 A1
(43) Pub. Date: Jan. 12, 2012

(54) DRUG CARRIER FOR TREATING OF GASTROINTESTINAL ULCER

(76) Inventors: Chih-Hui YANG, Chiayi City (TW); Keng-Shiang HUANG, Chiayi County (TW)

(21) Appl. No.: 12/832,673

(22) Filed: Jul. 8, 2010

(51) Int. Cl. A61K 9/14; A61P 1/04; A61K 31/4439

(52) U.S. Cl. 424/489; 514/338

(57) ABSTRACT

A drug carrier for treating of gastrointestinal ulcer comprises a microspheroid having a substrate and a plurality of particles distributed over and covered by the substrate, wherein the substrate is alginate and the plurality of particles is a drug for treating of gastrointestinal ulcer; and a coat covering up the surface of the microspheroid, which contains chitosan.

### BACKGROUND OF THE INVENTION

1. Field of the Invention

The present invention relates to a drug carrier, particularly to a drug carrier for treating of gastrointestinal ulcer.

2. Description of the Related Art

Gastrointestinal ulcer, also called peptic ulcer or UC is an ulcer generally occurred at gastrointestinal tract, which may lead to mucosal erosions, inflammatory, hemorrhaging and perforation usually accompanied with peptic damages and extremely pain. The gastrointestinal ulcer is a lifestyle disease mainly associated with chronic inflammatory due to psychological stress, infection of Helicobacter pylori, irregular diets and secretion disorders, especially for gastric acid, which is frequently appearing in some industrial countries.

According to the treatment of the gastrointestinal ulcer, patients with ulcer-like symptoms are often treated with antacids, histamine antagonist and protein pump inhibitor also called PPI. The antacids are mainly base or basic salt, primary for neutralizing stomach acidity, membrane protection and tissue reconstruction. Generally, treatment with antacids alone is mainly for symptomatic relieving especially only justified for minor symptom. The histamine antagonist is an agent for histamine inhibition, which is usually used for reducing excessive secretion of gastric acid and mucous inflammation. However, the systemic anti-histamine reaction of the histamine antagonist may lead to serious side effects to patients. The PPI is the most potent inhibitors of acid secretion available today, for providing a pronounced and long-lasting reduction of gastric acid production by irreversibly blocking the hydrogen/potassium adenosine triphosphatase enzyme system (also called H+/K+ATPase) of the gastric parietal cell. In this way, the proton pump of the gastric parietal cell may no longer secret H+ions into the gastric lumen so as to inhibit the secretion of gastric acid.

Although, PPI is a well-tolerated medication for treating of gastrointestinal ulcer, it has some inefficiency in pharmacokinetics such as inactivity while co-administration with food or potential side effects. In general, the absorption rate of proton pump inhibitor is decreased and delayed by concomitant food intakes, which may interfere with the therapeutic effects of PPI. Additional, due to the diversity of pharmacokinetics of PPI in bodies, some adverse effects include headache or nausea may occurred accompanying with long-term of treatment. In recent decades, according to the fast development of drug carrier, it is sufficient to provide a delivery system for the PPI in order to promote the therapeutic effects, also avoid the potential side effects as biomedical application.

For example, a conventional drug carrier as shown in Taiwan patent no. 200613011 discloses a nanoporous polymer, comprising a chitosan polymer encapsulated in surface modified colloidal nanoporous particles of silicon dioxide, wherein the chitosan polymer contains drugs treated of gastrointestinal ulcer for relieving of ulcerated symptoms, and chitosan, with biocompatibility and biodegradation for membrane adhesion and drugs sustainable releasing. Moreover, the encapsulated chitosan polymer and surface modified colloidal nanoporous particles of silicon dioxide are combined to form a condense structure which shows high stability in an acidic environment, in order to prolong the therapeutic effects of drugs in bodies.

Another conventional drug carrier for treating of gastrointestinal ulcer as disclosed in Taiwan patent no. 20070281015 comprises an sodium alginate, a PPI and at least one soluble or partially soluble antacid for suppressing the excess secretion of gastric acid. Wherein, the antacid in the conventional drug carrier is primary selected from a group of soluble or slightly soluble inorganic salt, such as calcium carbonate, sodium carbonate and aluminum hydroxide, for counteracting excess acidity in gastric stomach. On the other hand, the sodium alginate will perform at a plurality of colloidal suspension in stomach in order to carry out the encapsulation of the PPI for sustaining drugs releasing, also maintaining the mucosa membrane of stomach.

Furthermore, in Taiwan patent no. 464514 discloses the other drug carrier comprising a PPI, a mixture of at least one antacid and alginate and an excipient contained any other acceptable therapeutic integration within an oral medicinal form.

In summary, the conventional drug carriers as described above mainly contain a polymer, alginate for example, as a main substance of drug carrier to encapsulate a drug, PPI for example, in order to present with a sustainable releasing effect. However, a single coating construction of the polymer tends to be easily collapsed while suffering from the digestive process in bodies, especially under the erosion of gastric acid in stomach. In this situation, the sustainable releasing effect of the conventional drug carrier may be interrupted, and according the function and activity of the encapsulated drugs in drug carrier will become poor efficiency and less persistence. Additionally, although the conventional drug carrier disclosed in Taiwan patent no. 200613011 contains the chitosan polymer for membrane adhesion and directly treating of affected parts in gastrointestinal tract, the encapsulated structure between the chitosan polymer and surface modified colloidal nanoporous particles of silicon dioxide still have problems in sustainable drug releasing and structural maintenance under the process of gastric acid. Also, due to the safety issue of non-biodegraded materials generally used in the conventional drug carrier, the industrial application of the conventional drug carriers need further adjusting and modification for avoiding negative side effects involved in. Hence, there is a need of improving the disadvantages of the conventional drug carriers for treating of gastrointestinal ulcer.

### SUMMARY OF THE INVENTION

The primary objective of this invention is to provide a drug carrier for treating of gastrointestinal ulcer, which can improve the disadvantage of the conventional drug carrier, avoid the erosion by gastric acid, as well as maintain the sustainable releasing of drug.

The secondary objective of this invention is to provide a drug carrier for treating of gastrointestinal ulcer which can attach to the gastrointestinal tract in bodies for directly treating of the affected parts in order to promote the therapeutic efficiency of drugs.

Another objective of this invention is to provide a drug carrier for treating of gastrointestinal ulcer which is completely made from biological materials in order to avoid any unexpected side effects.

A drug carrier for treating of gastrointestinal ulcer comprises a microspheroid having a substrate and a plurality of particles distributed over and covered by the substrate, wherein the substrate is alginate and the plurality of particles is a drug for treating of gastrointestinal ulcer; and a coat covering up the surface of the microspheroid, which contains chitosan.

Further scope of the applicability of the present invention will become apparent from the detailed description given hereinafter. However, it should be understood that the detailed description and specific examples, while indicating preferable embodiments of the invention, are given by way of illustration only, since various will become apparent to those skilled in the art from this detailed description.

### BRIEF DESCRIPTION OF THE DRAWINGS

The present invention will become more fully understood from the detailed description given hereinbelow and the accompanying drawings which are given by way of illustration only, and thus are not limitative of the present invention, and wherein:

FIG. 1 is a diagram illustrating a structure of a drug carrier in the present invention;

FIG. 2 is a line chart illustrating a drug releasing phase in stomach in the present invention;

FIG. 3 is line chart illustrating a drug releasing phase in intestine in the present invention;

FIG. 4 is a diagram illustrating an electrospinning manufacturing process of the drug carrier according to the preferred embodiment in the present invention;

FIG. 5 is a bar chart illustrating a relation between the magnitude of electric field and the diameter of drug carrier for treating of gastrointestinal ulcer in the present invention;

SOLE PHOTO is an electron microscope photograph of the drug carrier for treating of gastrointestinal ulcer in the present invention

In the various figures of the drawings, the same numerals designate the same or similar parts.

### DETAILED DESCRIPTION OF THE INVENTION

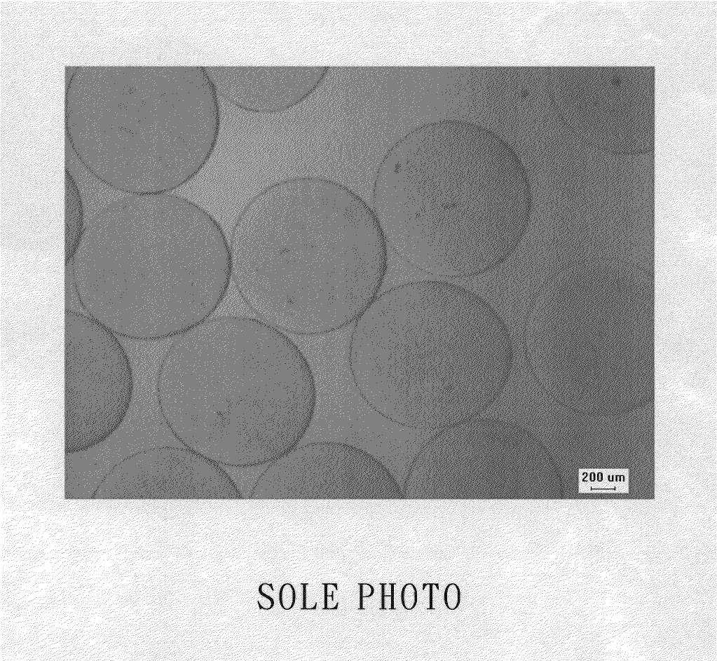

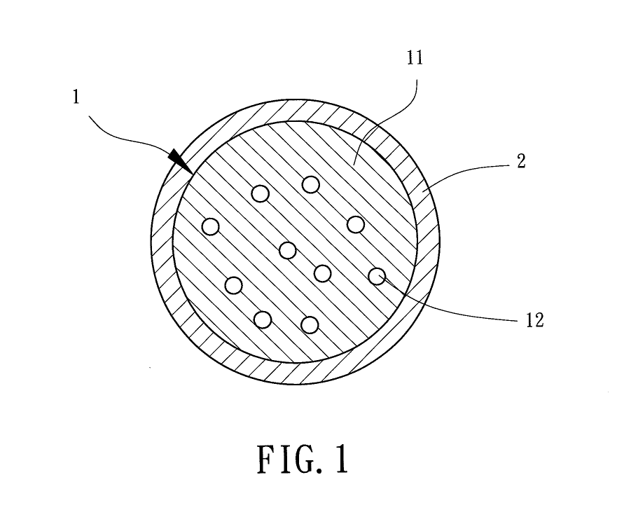

Referring to FIG. 1, the drug carrier for treating of gastrointestinal ulcer in the present invention comprises a microspheroid 1 including a substrate 11 and a plurality of particles 12 and a coat 2 for covering the microspheroid. In the present invention, the substrate 11 of the microspheroid 1 and the coat 2 are preferable to be made from a naturally biodegradable material, with ability of ionized and crosslinking-gelating reaction under a preferable environment. The plurality of particles is a drug, especially for treating of gastrointestinal ulcer, such as PPI, inhibitors and antacids.

In the preferred embodiment, the naturally biodegradable material used in the present invention is selected form a group of alginate, chitosan, collagen and other polymers, generally with a hydrolyzed bond, ester and carbamine for example, which can be self-degradable by enzymatic effects in bodies. In this situation, due to the biocompatibility and biodegradation of the naturally biodegradable material, it is preferable to apply to biomedical application, with predictability in biological response, reaction rate and possible toxin than synthetic polymers. Therefore, the drug carrier for treating of gastrointestinal ulcer in the present invention is safe in use.

The substrate 11 in the preferred embodiment can be alginate, a natural polysaccharide, which is soluble in saline presenting with negative charge, also with crosslink-gelating relation with divalute cation to perform at a plurality of colloidal particles for encapsulating the drug. In this way, theplurality of colloidal particles of alginate from crosslink-gelating relation is dramatically stabilized even in an acidic environment around pH 3.5, and accordingly the colloidal structure and sustainable releasing effect of the drug carrier in the present invention can be maintained in stomach.

The plurality of particles 12 is a protein pump inhibitor (PPI) selected from a group of omeprazole, lansoprazole, dexlansoprazole, esomeprazole, pantoprazole and rabeprazole, wherein, the PPI is selectively but specifically blocked the function of H/K ATPase in gastric cell for decreasing the secretion of the gastric acid. Additionally, the PPI is widely spread and encapsulated in the plurality of colloidal particles of alginate via the crosslinking-gelating reaction, so that the structure of the microspheroid 1 can be maintained in stomach followed by sequentially drug released and absorbed in intestine. Hence, the PPI in the drug carrier for treating of gastrointestinal ulcer can be sustainable released in a more efficient manner for longer persisting period so as to the therapeutic effects of the PPI can be well performed.

The coat 2 includes a chitosan, a polysaccharide, for providing muli-functions of encapsulation, membrane adhesion and hemostasis to the microspheroid 1, with biodegradation, bioavailability and antisepticised effect in bodies, also shows positive-charge under an acidic environment. In this way, due to the mult-functions of chitosan, the drug carrier can attach to the membrane of stomach for directly delivering the drug to affected part via the cation transmission in the acidic environment. Moreover, the cation of chitosan in the coat 2 and the negatron of alginate in the microspheroid 1 tents to perform at a strong binding relation between each other for stabilizing the structure of the microspheroid 1. Therefore, the drug carrier in the present invention may not be interfered by the process of stomach, but further prolong the persisting time in digestive tracts for sufficiently drug releasing.

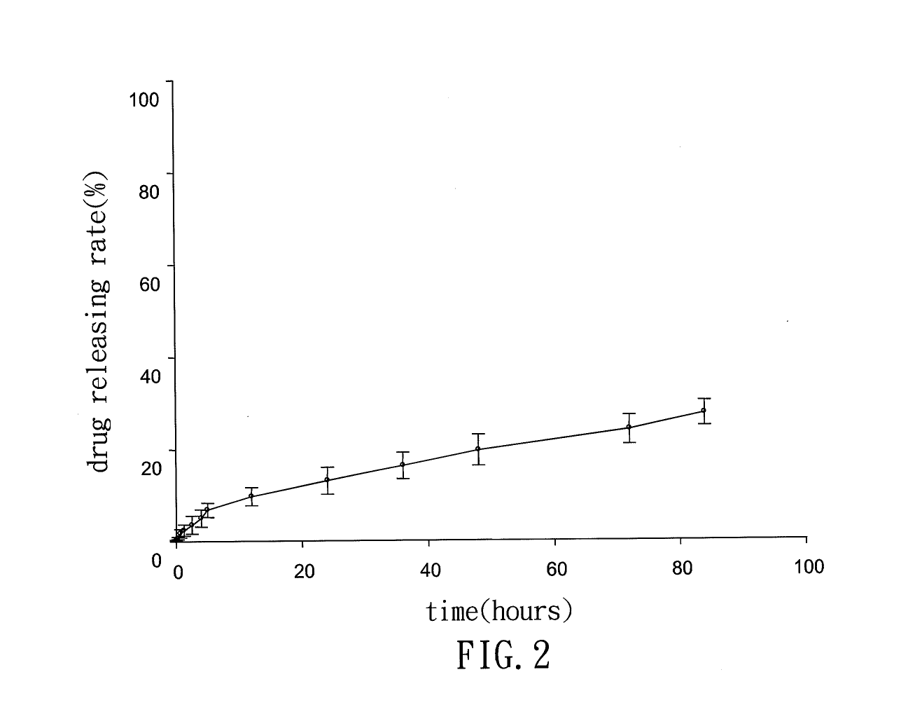

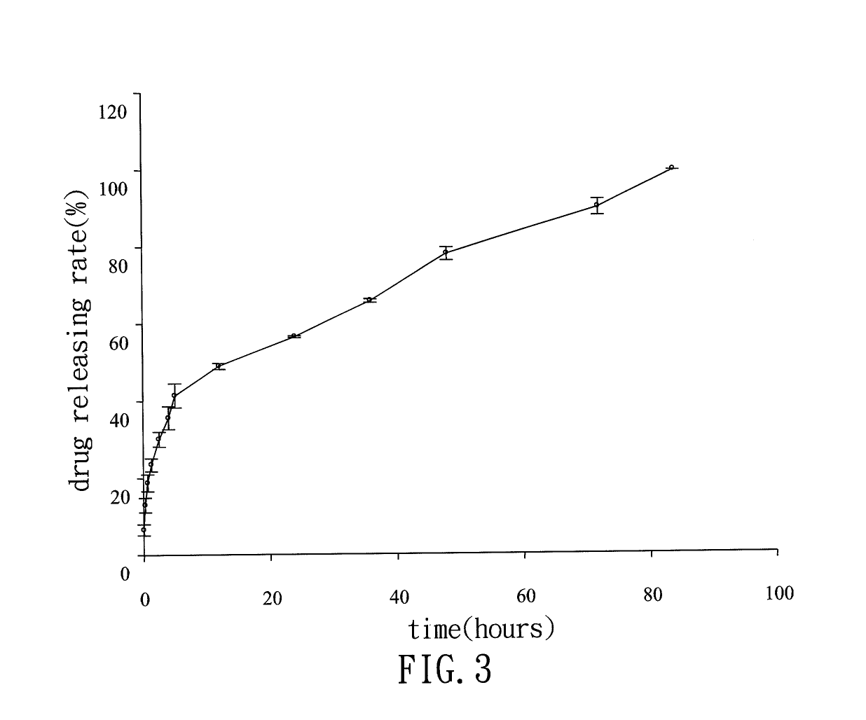

Referring to FIGS. 2 and 3, illustrates the drug releasing in stomach and intestine while the drug carrier in the present invention has delivered in bodies, wherein, only 20% to 30% of drug has released by times in the stomach (as shown in FIG. 2), however, more than 60% of drug can be released in the intestine in 24 hours (as shown in FIG. 3). In additional, the drug releasing rate has increased by times, with around 100% of drug releasing in 80 hours after delivery. It is suggested that, the drug carrier for treating of gastrointestinal ulcer in the present invention shows preferable structural maintenance to encapsulated drug even in gastric acid thus the sustainable drug release and absorption can be observed in the intestine of bodies. In this situation, the therapeutic effects of the encapsulated drug can be performed in a more efficient way.

For precisely description, in FIG. 2, while the drug carrier for treating of gastrointestinal ulcer has delivered to bodies, the drug carrier may attach to the cells of gastric membrane or upper intestine via the membrane adhesion of chitosan for directly applying the therapeutic effects of hemostasis and sterilization to affected parts. At the mean time, due to the penetration of the chitosan in acidic environment, the drug carrier has pierced through the gastric cells for directly delivering of drug to affected part. Hence, 20% to 30% of drug releasing has been observed at stomach, which indicates the adhesion, penetration and drug releasing of partial drug carriers in gastric cells. It is suggested that the drug carrier for treating of gastrointestinal ulcer in the present invention can attach to the gastric cells for directly treating of affected parts in order to promote the therapeutic efficiency of the drug for treating of gastrointestinal ulcer. On the other hand, the manufacture method of drug carrier for treating of gastrointestinal ulcer in the present invention is mainly categorized into free dropping, emulsion, spraying and electrospinning methods. As an example, the drug carrier in the preferred embodiment is obtained from the electrospinning method, with an electric field driving up the plurality of colloidal particles of substrate 11 (alginate) to encapsulate the plurality of particles 12 (the PPI) for resulting in microspheroid 1, also the coat 2 (chitosan) performed a strong binding relation with the microspheroid 1 to finish in two steps. In this way, the manufacturing process of the drug carrier in the present invention is simplified and well-controlled via the precise adjustment by the electric field.

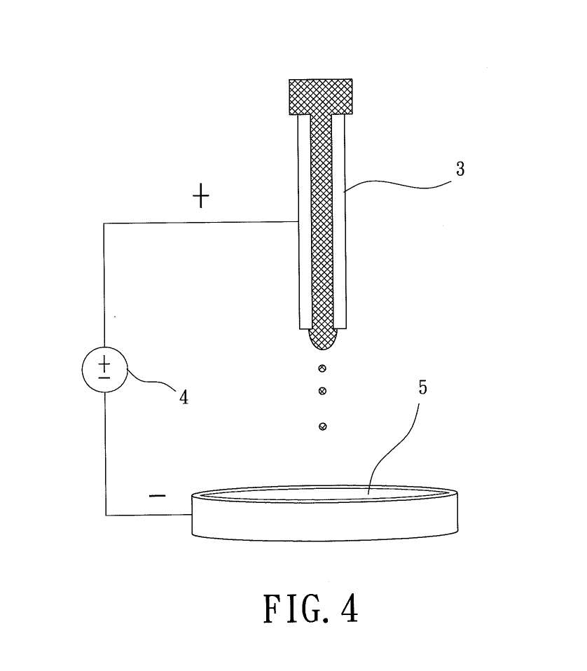

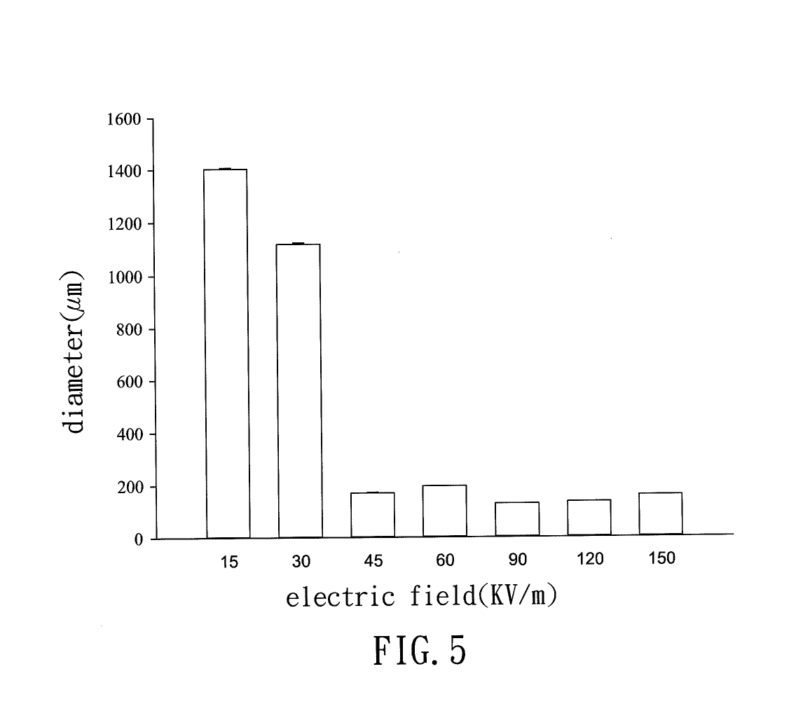

Referring to FIG. 4, illustrates the diagram of electrospinning method of the drug carrier in the present invention. In the preferred embodiment, a mixture 3 of 4% of alginate (as the substrate 11) and 40 mg/ml of PPI (as the plurality of particle 12) in a ratio of 1:1 is propelled by an electric field 4 for sequentially dropping into a crosslinking-gelating agent 5 contained 25% of calcium agent and 10% of chitosan in a ratio of 1:1 for obtaining the drug carrier under the crosslinking-gelating reaction between the calcium and alginate, calcium and chitosan, also alginate and the chitosan. As shown in the sole Photo, a plurality of drug carriers with uniform diameter and regular shape are obtained followed by the driving of the electric field. In Additional, based on the FIG. 5, when the magnitude of the electric field is diverse from 15 to 30 kilovoltage or from 45 to 150 kilovoltage for 1 milliliter per hour, the drug carriers distributed in various diameter may be obtained with approximately 1100 to 1500 micrometer (μm) or 120 to 200 μm of diameter.

Through the present invention, the drug carrier for treating of gastrointestinal ulcer contains alginate and chitosan which can provide a strong encapsulated structure under acid environment for stabilizing the composition and drug releasing effect as passing through the gastrointestinal tract. Furthermore, the chitosan in drug carrier has multifunction of membrane adhesion and hemostasis which can directly attach to the gastrointestinal tract for treating of affected parts in a more efficiency manner. On the other hand, the drug carrier is primary from biodegraded materials, such as alginate and chitosan, which is low in toxicity but high in bioavailability, biocompatibility, as well as safety. As a result, the drug carrier for treating of gastrointestinal ulcer in the present invention is significant for clinical gastrointestinal ulcer-related patients to provide a new drug delivering system with higher security, better therapeutic effects, also sustainable drug releasing performance as treatment.

Although the invention has been described in detail with reference to its presently preferred embodiment, it will be understood by one of ordinary skill in the art that various modifications can be made without departing from the spirit and the scope of the invention, as set forth in the appended claims.

What is claimed is:

1. A drug carrier for treating of gastrointestinal ulcer, comprising:
a microspheroid having a substrate and a plurality of particles distributed over and covered by the substrate, wherein the substrate is alginate and the plurality of particles is a drug for treating of gastrointestinal ulcer; and
a coat covering up the surface of the microspheroid, which contains chitosan.

2. The drug carrier for treating of gastrointestinal ulcer as defined in claim 1, wherein the drug for treating of gastrointestinal ulcer is a proton pump inhibitor.

3. The drug carrier for treating of gastrointestinal ulcer as defined in claim 2, wherein the proton pump inhibitor is selected from a group of omeprazole, iansoprazole, dexlansoprazole, esomeprazole, pantoprazole, esomeprazole pantoprazole and rabeprazole.

4. The drug carrier for treating of gastrointestinal ulcer as defined in claim 1, wherein the weight ratio between the substrate and the plurality of particles is 1:1.

5. The drug carrier for treating of gastrointestinal ulcer as defined in claim 1, wherein the weight ratio between the chitosan and microspheroid is 1:1.

6. The drug carrier for treating of gastrointestinal ulcer as defined in claim 1, wherein the diameter of the drug carrier is from 120 to 1500 micrometer (μm).

7. The drug carrier for treating of gastrointestinal ulcer as defined in claim 1, wherein the drug carrier is manufactured by at lease one of free dropping method, emulsion electrospinning method and spraying method.
